(19) United States
(12) Patent Application Publication
(10) Pub. No.: US 2012/0301913 A1
(43) Pub. Date: Nov. 29, 2012

(54) Microplate Assemblies for Flux Determination by Gradient Monitoring and Methods for Using Same

(76) Inventors: Cody Youngbull, Tempe, AZ (US); Kevin P. Timms, Tempe, AZ (US); Jakrey Myers, Scottsdale, AZ (US); Deirdre R. Meldrum, Phoenix, AZ (US)

(21) Appl. No.: 13/480,243

(22) Filed: May 24, 2012

Related U.S. Application Data
(60) Provisional application No. 61/489,647, filed on May 24, 2011.

(51) Int. Cl. C12M 1/34; G01N 21/64

(52) U.S. Cl. 435/29; 435/288.5

(57) ABSTRACT

Microplate assemblies and methods for using same are provided. In accordance with some embodiments, microplate assemblies are provided, the assemblies comprising a microplate well portion that forms a microplate well and that includes at least one sensor in the microplate well that responds to analyte in the well. In accordance with some embodiments, methods for determining a cell consumption rate of an analyte are provided, the methods comprising: positioning a cell in a microplate well having at least one sensor located within the well; providing analyte to the microplate well at a controlled rate; detecting a response of the sensor to the analyte in the microplate well; and determining a cell consumption rate of the analyte based on the response of the sensor to the analyte.

### CROSS REFERENCE TO RELATED APPLICATION

The application claims the benefit of U.S. Provisional Patent Application No. 61/489,647, filed May 24, 2011, which is hereby incorporated by reference herein in its entirety.

### TECHNICAL FIELD

The disclosed subject matter relates to microplate assemblies for flux determination by gradient monitoring and methods for using same.

### SUMMARY

Microplate assemblies for flux determination by gradient monitoring and methods for using same are provided. In accordance with some embodiments, microplate assemblies are provided, the assemblies comprising a microplate well portion that forms a microplate well and that includes at least one sensor in the microplate well that responds to analyte in the well. In accordance with some embodiments, methods for determining a cell consumption or production rate, also referred to as the flux, of an analyte are provided, the methods comprising: positioning a cell in a microplate well having at least one sensor located within the well; providing analyte to the microplate well at a controlled rate; detecting a response of the sensor to the analyte in the microplate well; and determining a cell consumption or production rate of the analyte based on the response of the sensor to the analyte. The flux follows from the gradient of analyte generated in the microwell.

### BRIEF DESCRIPTION OF THE DRAWINGS

FIG. 1 is a diagram of a microplate assembly portion in an opened position in accordance with some embodiments.

FIG. 2 is a diagram of a microplate assembly portion in a closed position in accordance with some embodiments.

FIG. 3 is a diagram of a microplate assembly portion having optical-capillary sensors in accordance with some embodiments.

FIG. 4 is a diagram of a microplate assembly portion having a multi-lumen-optical-capillary sensors in accordance with some embodiments.

### DETAILED DESCRIPTION

Microplate assemblies and methods for using same are provided.

In accordance with some embodiments, a cell consumption or production rate of an analyte can be detected by collecting a series of one or more luminescence (fluorescence or phosphorescence) images each taken within a medium of a microplate well.

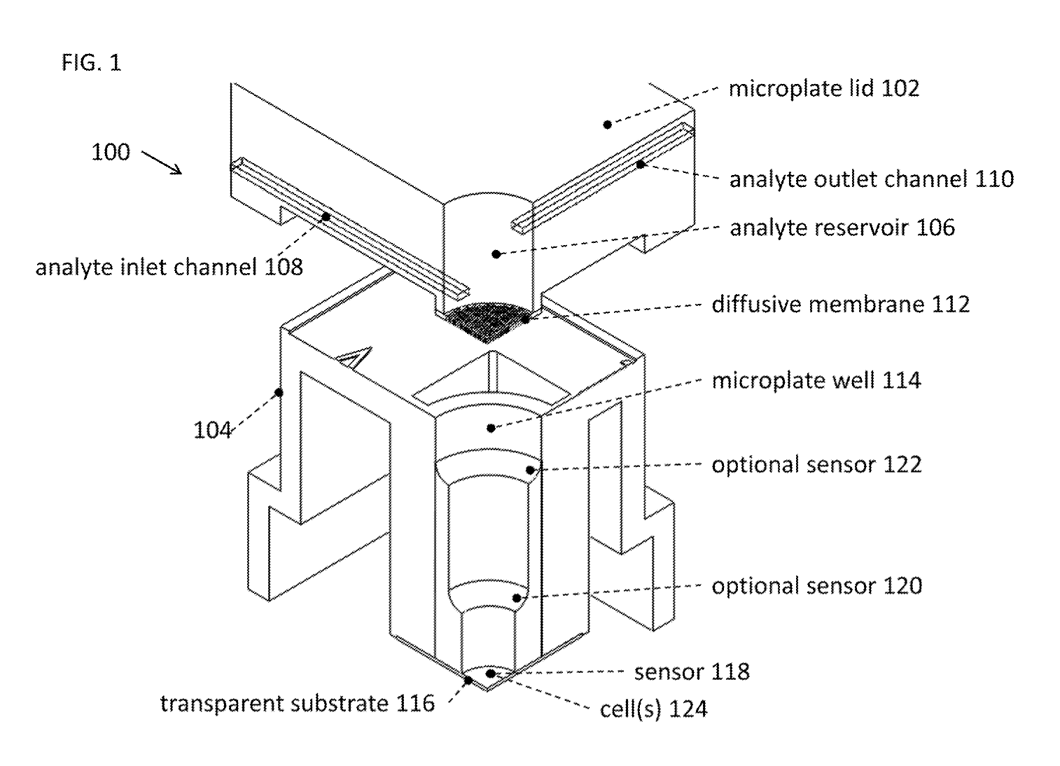

In accordance with some embodiments, a microplate assembly is provided. FIG. 1 illustrates an example of a portion 100 of a microplate assembly in accordance with some embodiments. As shown, portion 100 includes a microplate lid portion 102 and a mircoplate well portion 104. The microplate lid portion 102 can be removably placed on top of the microplate well portion 104, and, in some embodiments, as described below, microplate lid portion 102 can be omitted from the microplate assembly.

Portion 100 can be replicated any suitable number of times in the microplate assembly. For example, the microplate assembly can have 8, 16, 32, 64, etc. number of portions 100 arranged in any suitable fashion. Two or more of the microplate lid portions of the microplate assembly can be integrated into a single structure. Likewise, two or more of the microplate well portions of the microplate assembly can be integrated into a single structure.

As shown in FIG. 1, the microplate lid portion 102 can include an analyte reservoir 106, an analyte inlet channel 108, an analyte outlet channel 110, and a diffusive membrane 112. The microplate well portion 104 can include a microplate well 114, a transparent substrate 116 forming the bottom of the well, a first sensor 118, a second, optional sensor 120, and a third, optional sensor 122.

The analyte reservoir in the microplate lid can be filled with a known concentration of analyte in fluid via the analyte inlet channel at a desired rate. Fluid in the analyte reservoir can be removed via the analyte outlet channel at a desired rate. Thus, for example, an analyte-containing fluid in the analyte reservoir can be replenished at a fixed, known concentration via the analyte inlet and analyte outlet channels that run to each analyte reservoir in the microplate lid. Analyte in the reservoir can also exit the reservoir into the microplate well 114 via the diffusive membrane 112. Analyte which diffuses through the membrane can mix without creating appreciable turbulence into the microplate well 114 and thus can provide a known concentration of analyte at the membrane boundary independent of a cell consumption or production rate in the well. This known, fixed concentration along with one or more sensors inside microplate well 114 can be used to determine the analyte gradient within the well.

Microplate well 114 can have any suitable shape. Sensors 118, 120, and 122 can be any suitable sensors, such as fluorescent sensors that operate by optical fluorescent response (which may include changes in color, intensity or decay time of a fluorophore) in response to analyte.

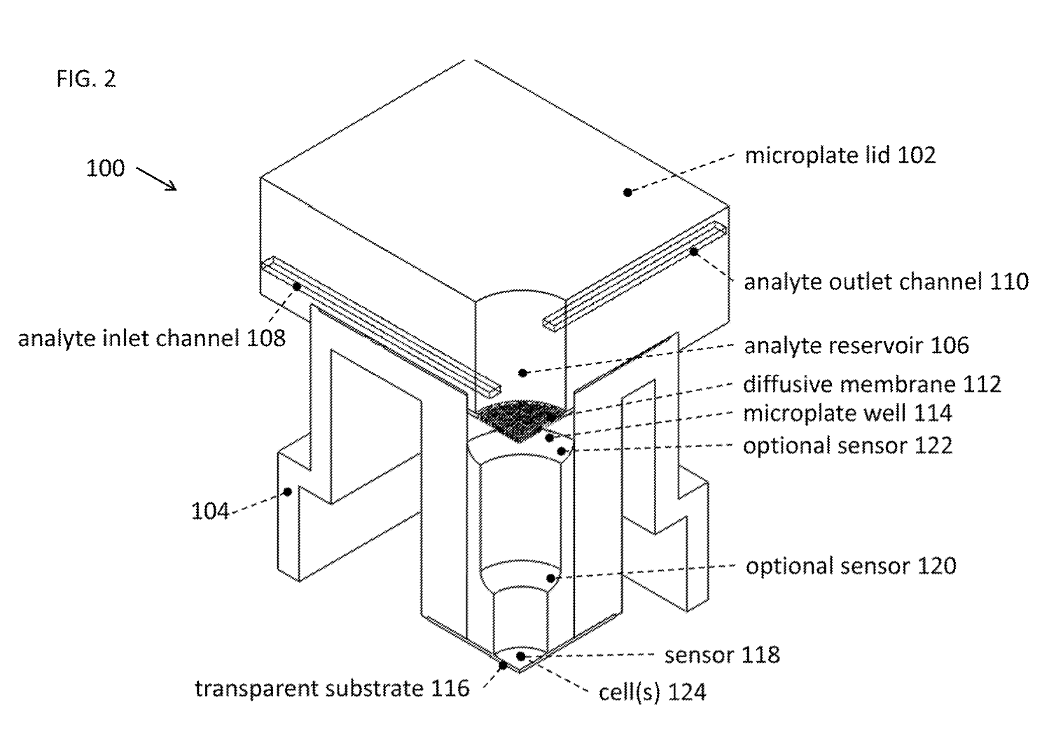

In some embodiments, first sensor 118 can be located at the plane of the cells at the bottom of the well, first optional sensor 120 can be placed at some intermediate height, and second optional sensor 122 can be placed just below the analyte reservoir. Although cells 124 and sensor 118 are illustrated as being in the same region at the bottom of the well in FIGS. 1 and 2, the cells and the sensor can be physically separated from each other or can be in contact with each other (e.g., such as the cells being located on a biocompatible thin film sensor on which the cells are growing).

Although three sensors 118, 120, and 122 are illustrated in FIG. 1, any suitable number of sensors (including only one) can be used in some embodiments, and those sensors can be at any suitable locations within the well.

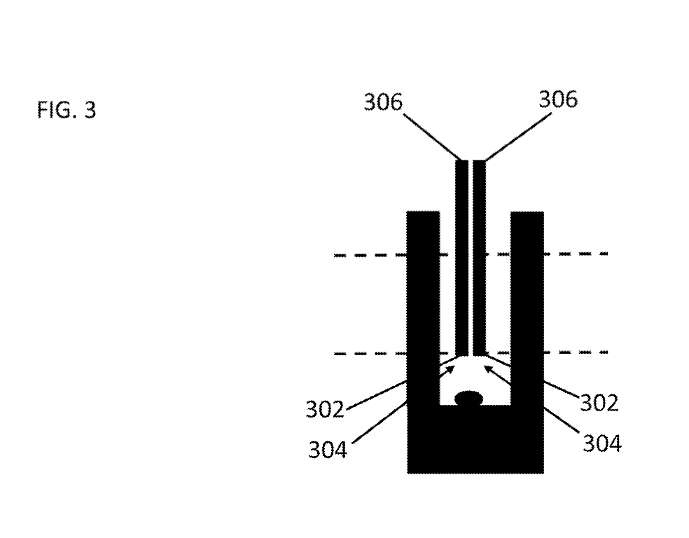

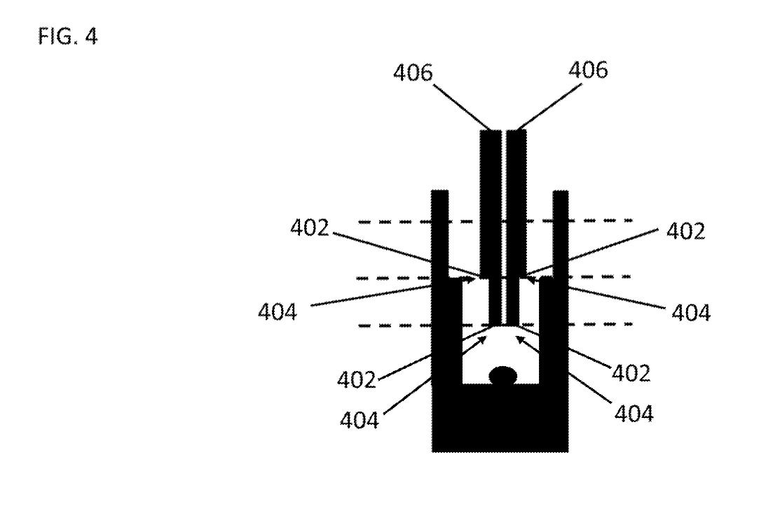

In some embodiments, rather that integrating one or more sensors with the walls of the microplate well, optical sensors can be provided as illustrated in FIGS. 3 and 4. As shown in FIG. 3, optical sensors 302 can be provided at the tips 304 of optical capillaries 306. Any suitable capillary, such as commercially available capillaries, can be used in some embodiments. Stimuli can be delivered through the central tube of the capillary and optical readout of the sensor can be achieved through the capillary wall. As shown in FIG. 4, optical sensors 302 can be provided at the tips 304 of multi-lumen optical capillaries 406 in order to provide sensors at different heights. Any suitable multi-lumen capillary, such as commercially available capillaries, can be used in some embodiments.

During use, one or more cells 124 can be placed in well 114 in any suitable fashion, such as at the bottom of the well on top of transparent substrate 116. The cells can then be exposed to analyte. Examples of analyte can include any soluble gas, ions, small molecules, nanoparticles, drugs, biomolecules, and microorganisms that reside at or below the lowest sensor location, for example cells 124 adhered to the transparent substrate 116 at the bottom of microplate well 114. Images of the sensors at or above this analyte plane can then be captured to measure the gradient of analyte in the microplate well 114 from which the analyte consumption rate can be determined.

In some embodiments, as mentioned above, delivery of analyte to the cells in the well can occur as the analyte permeate through the diffusive membrane of the lid portion. In some embodiments, if the analyte under study is a liquid borne analyte, and the lid is in place, the liquid borne analyte can be held at a constant value within the analyte reservoir by controlling the flow of analyte in channels 108 and 110. By controlling the concentration of analyte in the reservoir, the amount of analyte being delivered to the well can be controlled. In some embodiments, images of the fluorescent sensors can then be captured (e.g., from below microplate well via a suitable detector positioned below the transparent substrate).

As mentioned above, in some embodiments, microplate lid portion 102 can be omitted from the microplate assembly. For example, in some embodiments, if the analyte under study is a gas, the gas is present in the atmosphere, and a standard partial pressure is sufficient, then a microplate lid can be omitted from microplate portion 100. Without the microplate lid (possible for atmospheric equilibrating gases), an image of the fluorescent sensors can be captured from above the microplate well (because the lid is omitted) or below the microplate well (via the transparent substrate), for example.

Once steady state is reached between the two contributions to analyte concentration within the microplate well (i.e., the influx/efflux from the diffusive membrane and the opposite efflux/influx from the cell(s)), then, in accordance with Fick's Laws of diffusion, a linear gradient in the microplate well will exist. Because the concentration is known in the analyte reservoir, by measuring the analyte concentration at at least one position within the microplate well, the linear gradient slope of the analyte in the well, and hence the consumption rate, can be determined. In some embodiments, by measuring the slope at multiple positions, further confidence about the absolute consumption rate of the cell(s) can be determined.

The extracellular flux rate of an analyte in the steady state may be determined by observation of the gradient of its concentration. In the steady state, the analyte concentration gradient above cells in a well can be linear as seen below in Equation 3. It may therefore not be necessary to measure this gradient in the vicinity of the cells and thereby risk damaging them. Measuring the analyte concentration at different depths within the well can be used to verify the linearity of the gradient, and hence both validate the steady state assumption and calculate the gradient by simple linear regression of the observed concentration values against the position of the sensor. The sensor may be moved or multiple fixed sensors may be used.

Equations:

(1) Fick's second law

$$\frac{\partial C}{\partial t} = D \frac{{\partial }^{2} C}{{\partial }^{2} z}$$

(2) Steady state condition

$$D \frac{{\partial }^{2} C}{{\partial }^{2} z} = 0$$

(3) General solution to Eq. 1

C=az+b.

(4) Boundary conditions

C(h)=αP_{O}_{₂}_{,cells}; C(0)=αP_{O}_{₂}_{,surface}

(5) Specific solution to Eq. 1

$$C = \frac{α [ {P}_{{O}_{2 ,} cells} - {P}_{{O}_{2 ,} surface} ]}{h} \cdot z + α {P}_{{O}_{2 ,} cells}$$

(6) Fick's first law

$$J = - D \frac{\partial C}{\partial z}$$

(7) Combination of Eq. 5 and Eq. 6

$$J = - D \frac{α [ {P}_{{O}_{2 ,} cells} - {P}_{{O}_{2 ,} surface} ]}{h}$$

(8) Rate from flux

\dot{Q}=J·A

(9) Respiration rate from gradient

$$\dot{Q} = - ( D \cdot A \cdot α ) \frac{α [ {P}_{{O}_{2 ,} cells} - {P}_{{O}_{2 ,} surface} ]}{h}$$

### DEFINITION OF TERMS

C Concentration of Analyte (O₂, pH, . . . ) in units of mol/m³
t Time in units of s
z Position. Depth below surface of media column above the cells in units of m
D Diffusion coefficient (material constant) in units of m²/s
a,b Constants to be determined by boundary conditions
h Height of media column above the cells in units of m
α Analyte solubility coefficient. O₂ in media at 37° and standard pressure is reported as 0.94
P_{O}_{₂}_{,depth} Partial pressure of analyte in units of millimeters of mercury.
J Diffusion flux in units of mol/m².s
A Area of media column above the cell(s) in units of m²
\dot{Q} Rate of analyte consumption or production. The respiration rate of the cell(s) in units of mole/s

In accordance with some embodiments, this technique can be used to measure atmospheric equilibrated dissolved oxygen consumption rates. In some embodiments, this technique can additionally or alternatively be used to measure the differences in cell consumption rates between cell types (cancerous vs. normal) and also between differing growth morphologies on 2D planar untreated surfaces vs. treated surfaces (poly-L-lysine, fibronectin, and laminin) and 3D scaffolds of aligimatrix.

Any suitable mechanism for capturing images of the sensors can be used, such as a plate reader, an imaging cytometer, an optical microscope, etc. Through the sensor images, homogeneous fluid analyte consumption rate can be measured.

For example, in some embodiments, the consumption rate can be measured from a single image by determining the difference in the brightness of two sensors at different heights in the well, dividing by their height difference, and multiplying by constants. Imaging the number of cells in each well can be used in some embodiments to bin the responses according to isolated cell numbers and thus cell-cell interactions can be considered.

In some embodiments, there may be extracellular fluxes for which the concentration is so small that the approach above is not capable of measuring a response. In such situations, optical sensors can be placed in direct contact with light-guiding fibers coupled to high sensitivity photo-counting diodes. In this approach, images are not being acquired, but rather light levels of the sensors are being acquired, from which images can be inferred. Conversely, light levels can be inferred from images with appropriate calibration.

For example, in some embodiments, real time in situ control with a moving light-guiding capillary can be used. In such an approach, a specialized lid with multiple light-guiding capillaries attached can be placed over a microwell array. Next, the array can be placed in an imager (e.g., such as an upright fluorescence microscope, an imaging cytometer, a modified microarray plate scanner, etc.). Stimuli can then be introduced at will. Excess media can be allowed to drain so that its level is less than or equal to height of mircowell. Then, one can wait for cell response, which can take minutes in some embodiments. The lid can then be slowly raised and images periodically taken. Finally, the change in sensor brightness between images can be measured and fit by linear regression to determine analyte flux rate.

For example, in some embodiments, real time in situ control with a static multi-lumen light-guiding capillary can be used. In such an approach, a specialized lid with multiple light-guiding capillaries attached can be placed over a microwell array. Next, the array can be placed in an imager (e.g., an upright fluorescence microscope, an imaging cytometer, a modified microplate scanner, etc.). Stimuli can then be introduced at will. Excess media can be allowed to drain so that its level is less than or equal to height of mircowell. Then, one can wait for cell response, which can take minutes in some embodiments. The difference in photon counts from the light-guiding capillaries can be determined and fit by linear regression to determine analyte flux rate.

In some embodiments, the microwells and sensors can be produced by standard high throughput MEMS-type manufacturing. Any suitable technology can be used. For example, in some embodiments, one or more of the following technologies can be used to produce the microwells: replicate molding or soft lithography; repeated aligned photomasked lithography; reactive ion etching; etc. The diffusion constants of the microenvironment in the microplate well 114 should be dominated by the media diffusion, and not diffusion through surrounding materials microplate 104 and diffusive membrane 112. In the case of soft lithography with absorptive or emissive porous polymers, an additional coating might be required to ensure this condition. Sensors can be deposited by either additional photolithography or vapor phase deposition followed by barrier lift-off.

In some embodiments, any suitable solid phase sensor can be used. For example, hydrogel non-leeching fluorescent sensors for O₂ and pH as published in “SENSORS AND ACTUATORS B-CHEMICAL,” volume 147, issue 2, pages 714-722, Jun. 3, 2010, which is hereby incorporated by reference herein in its entirety, can be used in some embodiments. In some embodiments, these sensors can be mixed into a single matrix so long as their absorption and emission are compatible with the optical detection approach. In some embodiments, the sensitivity of these embedded sensors can be enhanced by resonant photonic behavior, for example, these sensors may act like micro-ring resonators which would be capable of measuring single-molecule changes.

In some embodiments, any suitable capillaries can be used. For example, in some embodiments, light-guiding capillaries, such as light-guiding fused silica capillaries, can be used. Light-guiding fused silica capillaries are commercially available, for example, from Polymicro Technologies, http://www.polymicro.com. Capillaries are available with dimensions appropriate for narrow single-cell appropriate microwells. Furthermore, published methods and commercial fittings exist for connecting both fluids and excitation/detection to these optical capillaries. In some embodiments, these capillaries can be prepared by dipping a pressurized (to keep the central channel free of sensor material) capillary tip into the appropriate sensor material, followed by curing.

In some embodiments, the methods and/or processes described herein for flux determination by gradient monitoring can be performed using any suitable devices. For example, in some embodiments, one or more computers, optical detectors/cameras/imagers, fluid controllers, robotic arms, and/or any other suitable devices can be used to place cells into a well, control the exposure of the cells to analyte, capture images of the sensors, and/or perform the calculations of flux, gradient, etc. A computer used for such purpose can be any of a general purpose device such as a computer or a special purpose device such as a client, a server, controller, etc. Any of these general or special purpose devices can include any suitable components such as a processor (which can be a microprocessor, digital signal processor, a controller, etc.), memory, communication interfaces, display controllers, input devices, etc.

In some embodiments, any suitable computer readable media can be used for storing instructions for performing the processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Although the invention has been described and illustrated in the foregoing illustrative embodiments, it is understood that the present disclosure has been made only by way of example, and that numerous changes in the details of implementation of the invention can be made without departing from the spirit and scope of the invention, which is only limited by the claim which follows. Features of the disclosed embodiments can be combined and rearranged in various ways.

What is claimed is:

1. A microplate assembly comprising a microplate well portion that forms a microplate well and that includes at least one sensor in the microplate well that responds to analyte in the well.

2. The microplate assembly of claim 1, further comprising a microplate lid portion that forms a reservoir and that includes a diffusive membrane that allows analyte to pass from the reservoir to the microplate well when the microplate lid portion is positioned on top of the microplate well portion.

3. The microplate assembly of claim 2, wherein the microplate lid portion further comprises an analyte inlet channel and an analyte outlet channel.

4. The microplate assembly of claim 1, wherein the at least one sensor is a fluorescent sensor.

5. The microplate assembly of claim 1, wherein the at least one sensor includes at least two sensors each positioned at a different height in the microplate well.

6. The microplate assembly of claim 1, further comprising a transparent substrate at the bottom of the microplate well.

7. A method for determining a cell consumption rate of an analyte comprising:
positioning a cell in a microplate well having at least one sensor located within the well;
providing analyte to the microplate well at a controlled rate;
detecting a response of the sensor to the analyte in the microplate well; and
determining a cell consumption rate of the analyte based on the response of the sensor to the analyte.

8. The method of claim 7, wherein the at least one sensor is a fluorescent sensor.

9. The method of claim 7, wherein the at least one sensor includes at least two sensors each positioned at a different height in the microplate well.

10. The method of claim 7, wherein detecting the response of the sensor comprises capturing an image of fluorescence of the sensor.

11. The method of claim 7, wherein the cell consumption rate is determined according to Fick's Law.

12. The method of claim 7, wherein the analyte is provided to the microplate well by allowing the analyte to permeate through a diffusive membrane of a microplate lid that is positioned above the microplate well.
